(19) United States
(12) Patent Application Publication
(10) Pub. No.: US 2010/0191051 A1
(43) Pub. Date: Jul. 29, 2010

(54) MEDICAL APPARATUS

(76) Inventors: Kiyoshi MIYAKE, Niiza-shi (JP); Ryohei Namba, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/754,088

(22) Filed: Apr. 5, 2010

Related U.S. Application Data
(63) Continuation of application No. PCT/JP2007/071035, filed on Oct. 29, 2007.

(51) Int. Cl. A61B 1/00

(52) U.S. Cl. 600/104

(57) ABSTRACT

A medical apparatus includes a first medical equipment, a second medical equipment, an indicator and a keeping portion. The first medical equipment is used for performing a medical action with respect to an object. The second medical equipment includes a coupling portion attachable to/detachable from the first medical equipment. The indicator is attached to the first medical equipment and/or second medical equipment. The indicator is configured to indicate that the first and second medical equipments are coupled with each other, simultaneous with coupling between the first and second medical equipments. The keeping portion irreversibly keeps a state indicated by the indicator.

### CROSS REFERENCE TO RELATED APPLICATIONS

This is a Continuation Application of PCT Application No. PCT/JP2007/071035, filed Oct. 29, 2007, which was published under PCT Article 21(2) in Japanese.

### BACKGROUND OF THE INVENTION

1. Field of the Invention

The present invention relates to a medical apparatus that couples a plurality of medical equipments to use them.

2. Description of the Related Art

For example, JP-A 2006-34573 (KOKAI) discloses an endoscopic light source system. The light source system is controlled in such a manner that a lamp of the light source system is turned on when a connector of an endoscope is coupled with a connector of the light source system, and it is controlled in such a manner that the lamp is turned off when the connectors are decoupled.

Applying a relationship between the connector of the endoscope and the connector of the light source system to a coupling portion of a medical equipment such as a disposable insertion portion of the endoscope and a coupling portion of the medical equipment such as a reusable operation portion, any means indicates a situation that the coupling portions of the insertion portion and the operation portion are coupled with each other in accordance with lighting of the lamp in a state that both the members are coupled with each other.

### BRIEF SUMMARY OF THE INVENTION

According to an aspect of the present invention, there is provided a medical apparatus includes: a first medical equipment used for performing a medical action with respect to an object; a second medical equipment including a coupling portion attachable to/detachable from the first medical equipment; an indicator attached to the first medical equipment and/or second medical equipment and configured to indicate that the first and second medical equipments are coupled with each other, simultaneous with coupling between the first and second medical equipments; and a keeping portion which irreversibly keeps a state indicated by the indicator.

### BRIEF DESCRIPTION OF THE SEVERAL VIEWS OF THE DRAWING

FIG. 1 is a schematic view showing an endoscopic system according to first to seventh embodiments;

FIG. 2A is a schematic cross-sectional view showing a separated state before a first coupling portion on an insertion portion side of an endoscope is coupled with a second coupling portion on an operation portion side of the endoscope in the endoscopic system according to the first embodiment;

FIG. 2B is a schematic cross-sectional view showing a state that the first coupling portion on the insertion portion side of the endoscope is coupled with the second coupling portion on the operation portion side of the endoscope in the endoscopic system according to the first embodiment;

FIG. 3 is a schematic cross-sectional view showing a separated state before the first coupling portion on the insertion portion side of the endoscope is coupled with the second coupling portion on the operation portion side of the endoscope in the endoscopic system according to a modification of the first embodiment;

FIG. 4A is a schematic cross-sectional view showing a separated state before the first coupling portion on the insertion portion side of the endoscope is coupled with the second coupling portion on the operation portion side of the endoscope in the endoscopic system according to a second embodiment;

FIG. 4B is a schematic cross-sectional view showing a state that the first coupling portion on the insertion portion side of the endoscope is coupled with the second coupling portion on the operation portion side of the endoscope in the endoscopic system according to the second embodiment;

FIG. 4C is a schematic front view showing a flat plate which moves and uses the first coupling portion on the insertion portion side of the endoscope to indicate that the first and second coupling portions are coupled when the first coupling portion on the insertion portion is coupled with the second coupling portion on the operation portion side of the endoscope in the endoscopic system according to the second embodiment;

FIG. 5 is a schematic cross-sectional view showing a separated state before the first coupling portion on the insertion portion side of the endoscope is coupled with the second coupling portion on the operation portion side of the endoscope in the endoscopic system according to a modification of the second embodiment;

FIG. 6 is a schematic view showing the inside of the endoscope which is in a state that the first coupling portion on the insertion portion side of the endoscope is coupled with the second coupling portion on the operation portion side of the endoscope in the endoscopic system according to a third embodiment;

FIG. 7 is a schematic cross-sectional view showing a part of the first coupling portion of the insertion portion of the endoscope which is in the separated state before the first coupling portion on the insertion portion side of the endoscope is coupled with the second coupling portion on the operation portion side of the endoscope in the endoscopic system according to the third embodiment;

FIG. 8 is a schematic cross-sectional view showing an oxygen indicator fixed to an air supply tube arranged in the first coupling portion on the insertion portion side in air supply tubes of the endoscope in the endoscopic system according to a fourth embodiment;

FIG. 9 is a schematic view showing the inside of the endoscope which is in a state that the first coupling portion on the insertion portion side of the endoscope is coupled with the second coupling portion on the operation portion side of the endoscope in the endoscopic system according to a fifth embodiment;

FIG. 10 is a schematic cross-sectional view showing a pH indicator fixed to a suction tube arranged in the first coupling portion on the insertion portion side in suction tubes of the endoscope in the endoscopic system according to the fifth embodiment;

FIG. 11A is a schematic view showing an outline configuration of digital paper arranged with the first coupling portion on the insertion portion side of the endoscope in the endoscopic system according to a sixth embodiment;

FIG. 11B is a schematic view showing a state that a voltage is applied to a microcapsule having black and white fine particles of the digital paper to separate the black and white fine particles from each other;

FIG. 12 is a schematic view showing the indication of the digital paper of the first coupling portion before and after the first coupling portion on the insertion portion side of the endoscope is coupled with the second coupling portion on the operation portion side of the endoscope in the endoscopic system according to the sixth embodiment;

FIG. 13 is a schematic view showing the endoscopic system according to a seventh embodiment;

FIG. 14A is a partial cross-sectional view which is an enlarged view of a part indicated by reference character 14A in FIG. 13, showing a state that a pigment-containing capsule is arranged in a pigment accommodation portion of a universal cable of the endoscope and the portion is sealed by a flexible transparent window in the state; and

FIG. 14B is a schematic cross-sectional view showing a state that the transparent window of the universal cable of the endoscope is pushed by the gravity of the endoscope and the pigment-containing capsule arranged in the pigment accommodation portion is destroyed to attach a pigment to an entire surface of the transparent window on the pigment accommodation portion side when the endoscope is put on a scope hanger of a trolley.

### DETAILED DESCRIPTION OF THE INVENTION

The best mode for carrying out the present invention will now be described hereinafter with reference to the drawings.

### First Embodiment

A first embodiment will be first explained with reference to FIGS. 1 to 3.

As shown in FIG. 1, an endoscopic system (a medical apparatus) 10 according to the embodiment includes an endoscope 12, a light source 14 that provides the endoscope 12 with light, a controller 16 that controls the endoscope 12, and a monitor 18 that displays an observation image.

The endoscope 12 includes an elongated insertion portion (a first medical equipment) 22 that is inserted into a body cavity and an operation portion (a second medical equipment) 24 arranged at a proximal end portion of the insertion portion 22. Of these portions, the insertion portion 22 is a disposable portion, and the operation portion 24 is a reusable portion that can be reused through cleaning, antisepsis, and sterilization. Of course, it is also preferable to configure the operation portion 24 as a disposable portion or adopt a configuration obtained by integrally forming the entire endoscope 12 as a disposable portion. In this embodiment, the insertion portion 22 is a disposable portion and the operation portion 24 is a reuse portion.

The insertion portion 22 sequentially includes a distal end hard portion 32, a bending portion 34, a flexible portion 36, and a first coupling portion 38 from a distal end side toward a proximal end side.

The distal end hard portion 32 is formed of a hard member. The distal end hard portion 32 is arranged at a distal end of the insertion portion 22. The bending portion 34 that can bend in, e.g., two or four directions is arranged at a proximal end portion of the distal end hard portion 32. The flexible portion 36 that can bend in accordance with a shape of the body cavity or the like is arranged at a proximal end portion of the bending portion 34. The distal end hard portion 32, the bending portion 34, and the flexible portion 36 are arranged on the same axis. Further, the first coupling portion 38 that is coupled with the operation portion 24 is arranged at a proximal end portion of the flexible portion 36.

The operation portion 24 includes an operation portion main body 42 having a second coupling portion 44, a universal cable 46 having one end connected with the operation portion main body 42, and a connector 48 arranged at the other end of the universal cable 46. The connector 48 can be attached to or detached from the light source 14 and the controller 16.

Further, the first coupling portion 38 of the insertion portion 22 and the second coupling portion 44 of the operation portion 24 can be attached to or detached from each other.

It is to be noted that built-in members such as a bending operation wire, an air supply tube, a water supply tube, a suction tube, an illumination optical system and an observation optical system, and others (which are not illustrated) are arranged in the insertion portion 22 and the operation portion 24. Further, when the first coupling portion 38 of the insertion portion 22 is attached to or detached from the second coupling portion 44 of the operation portion 24, the built-in members on the insertion portion 22 side are attached to or detached from the built-in members on the operation portion 24 side in, e.g., a mechanical and optical manner. Since the built-in members on the insertion portion 22 side are mechanically and optically connected to the built-in members on the operation portion 24 side, the first coupling portion 38 of the insertion portion 22 is coupled with the second coupling portion 44 of the operation portion 24 in a predetermined state by various means when coupling these portions. In other words, at the time of coupling the first coupling portion 38 of the insertion portion 22 with the second coupling portion 44 of the operation portion 24, various kinds of coupling means are allowed if these coupling portions are coupled in a predetermined state. Therefore, when coupling the first coupling portion 38 with the second coupling portion 44 is achieved, both the first coupling portion 38 and the second coupling portion 44 are prevented from being moved in an axial direction and a circumferential direction.

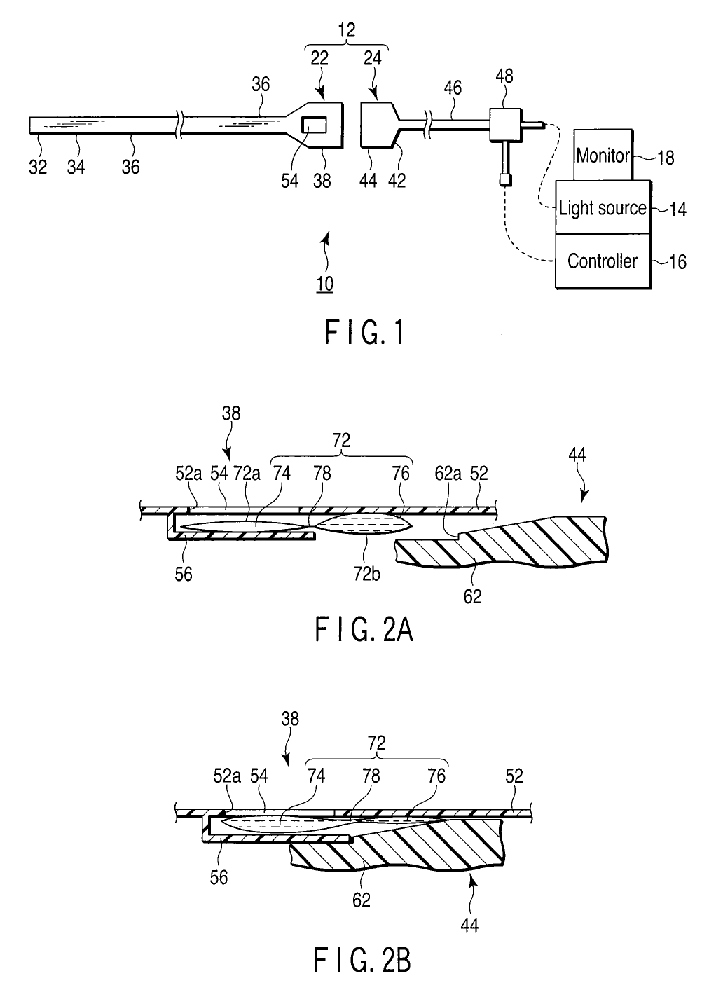

As shown in FIGS. 2A and 2B, the first coupling portion 38 of the insertion portion 22 includes an exterior frame 52 having an opening portion 52a, a window portion 54, and an accommodation portion 56. The exterior frame 52 can be attached to or detached from a later-described engagement portion 62 of the second coupling portion 44 of the operation portion 24.

The window portion 54 is arranged on the opening portion 52a of the exterior frame 52. The window portion 54 is transparent or translucent, and the inside can be observed through the window portion 54. The accommodation portion 56 is a portion where a later-described pigment-containing bag 72 is accommodated. The accommodation portion 56 is formed into a rectangular parallelepiped having a substantially-L-shaped vertical cross section and one opened surface. Further, the accommodation portion 56 is integrally formed with the exterior frame 52 and or fixed on the same by screwing and the like. At this time, the opening portion 52a of the exterior frame 52 or the window portion 54 faces the largest wall surface of the accommodation portion 56. Incidentally, it is preferable for a space between the exterior frame 52 and the accommodation portion 56 (a space between the window portion 54 and the largest wall surface of the accommodation portion 56) to be narrowly formed so that a pigment-containing bag 72 hardly moves away from the accommodation portion 56 when a pigment flows into a first chamber 74 of the later-described pigment-containing bag 72.

Therefore, the later-described first chamber 74 of the pigment-containing bag 72 is arranged in the window portion 54, and a later-described second chamber 76 is fixed to the exterior frame 52 by, e.g., an adhesive. Therefore, positions of the first chamber 74 and the second chamber 76 themselves of the pigment-containing bag 72 are prevented from being moved as much as possible.

The second coupling portion 44 of the operation portion 24 includes the engagement portion 62 that can be attached to or detached from the exterior frame 52 of the first coupling portion 38 of the insertion portion 22. A part of the engagement portion 62 is formed into a tapered shape so that the later-described second chamber 76 of the later-described pigment-containing bag 72 is pushed between the engagement portion 62 and the exterior frame 52 at the time of coupling the first coupling portion 38 of the insertion portion 22 with the second coupling portion 44 of the operation portion 24. At this time, a small space is formed in such a manner that a pushed state of the second chamber 76 is held between the engagement portion 62 and the inner peripheral surface of the exterior frame 52 after the pigment-containing bag 72 is pushed. Furthermore, a step 62a is formed at a position facing an end part of the accommodation portion 56.

As a material forming the pigment-containing bag 72, for example, a transparent and colorless film in which a polyethylene-based material and a polypropylene-based material are mixed is used. Rim parts of a front film 72a and a rear film 72b are strongly welded, and the pigment-containing bag 72 is divided into the first chamber (an empty compartment) 74 and the second chamber (a pigment-containing compartment) 76 by an easy-peel portion 78. The easy-peel portion 78 is weakly welded to block flow of fluid (the pigment) between the first chamber 74 and the second chamber 76. When the first chamber 74 or the second chamber 76 is pushed, the easy-peel portion 78 enters a communicating state, thereby readily achieving communication between the first chamber 74 and the second chamber 76.

Furthermore, the pigment such as iodine, congo red, phenol red, methylene blue, toluidine blue, indigo carmine, brilliant blue, or evans blue is put in the second chamber 76 of the pigment-containing bag 72. Moreover, in the pigment-containing bag 72, although a color of the pigment in the second chamber 76 containing the pigment can be visually confirmed, the first chamber 74 and the easy-peel portion 78 are transparent and colorless. As such a pigment-containing bag 72, using a double bag (Japanese Patent No. 3016348) having substantially the same configuration as PLW (a registered trademark) of Nipro Corporation is preferable, for example.

It is to be noted that, as the pigment, using one that meets criteria such as a Japanese Pharmaceutical Excipients or Japanese Standards of Food Additives is preferable. Additionally, the pigment of any color can be used as long as it is a color that readily allows recognition that the pigment has flowed into the first chamber 74 via the easy-peel portion 78 from the second chamber 76 through the opening portion 52a or the window portion 54. That is, it is preferable for the pigment to have a color that can be readily recognized as being largely different from a wall color of the accommodation portion 56 that is visually confirmed though the transparent first chamber 74. To compare colors in this manner, fixing a non-illustrated color comparison table to an edge part of the opening portion 52a of the exterior frame 52 in the first coupling portion 38 of the insertion portion 22 is preferable. The comparison table shows a correct color that is visually confirmed through the opening portion 52a or the window portion 54 before use and a correct color that is visually confirmed through the opening portion 52a or the window portion 54 after the insertion portion 22 is coupled with the operation portion 24 or after the endoscope 12 is used as will be described later. Therefore, when a user of the endoscope 12 compares the colors, whether the insertion portion 22 has been used can be easily recognized.

Further, as the pigment, it is preferable to use a pigment (e.g., iodine) that is not easily faded by cleaning when the pigment-containing bag 72 gets broken and the pigment adheres to the insertion portion 22 and/or the operation portion 24 of the endoscope 12 at the time of or after coupling the insertion portion 22 with the operation portion 24. Therefore, it can be easily recognized that the insertion portion 22 has been already connected with the operation portion 24.

Furthermore, a configuration that the window portion 54 is not provided on the opening portion 52a of the exterior frame 52 and the opening portion 52a alone is formed is also preferable.

A function of the endoscope 12 according to the embodiment will now be described.

As shown in FIG. 2A, when using the endoscope 12, the first coupling portion 38 of the insertion portion 22 is caused to face the second coupling portion 44 of the operation portion 24. At this time, the pigment is provided in the second chamber 76 of the pigment-containing bag 72 alone, and the first chamber 74 is clear and colorless. Therefore, a color of the wall surface of the accommodation portion 56 can be visually confirmed through the window portion 54 and the first chamber 74.

Furthermore, the first coupling portion 38 of the insertion portion 22 is coupled with the second coupling portion 44 of the operation portion 24 in a predetermined state. At this time, the engagement portion 62 of the second coupling portion 44 of the operation portion 24 is utilized to push the second chamber 76 of the pigment-containing bag 72. Therefore, the sealing of the easy-peel portion 78 is released by pressure provided at this moment, thereby achieving communication between the first chamber 74 and the second chamber 76 through the tubular easy-peel portion 78. Therefore, the pigment flows into the first chamber 74 from the second chamber 76 through the easy-peel portion 78 to fill the first chamber 74 with the pigment. Accordingly, the color of the pigment in the first chamber 74 can be visually confirmed through the window portion 54.

Therefore, it is recognized that the disposable insertion portion 22 is coupled with the reusable operation portion 24. The endoscope 12 in this state is used for, e.g., a living tissue.

Moreover, after use of the endoscope 12, the insertion portion 22 is separated from the operation portion 24. At this time, the pressing force of the engagement portion 62 in the second coupling portion 44 of the operation portion 24 with respect to the second chamber 76 of the pigment-containing bag 72 is released. However, the first chamber 74 of the front film 72a communicates with the second chamber 76 of the rear film 72b through the easy-peel portion 78, and then the pressing force for the second chamber 76 of the rear film 72b is gradually weakened with time. Therefore, the easy-peel portion 78 between the front film 72a and the rear film 72b is again closed. That is, a communication path between the first chamber 74 and the second chamber 76 is narrowed. Therefore, all of the pigment that has flowed into the first chamber 74 of the pigment-containing bag 72 does not return to the second chamber 76. Even if returning the pigment that has flowed into the first chamber 74 to the second chamber is tried, the pigment partially remains in the first chamber 74. Therefore, even though the coupling between the insertion portion 22 and the operation portion 24 is released, the state that the insertion portion 22 is being coupled with the operation portion 24 is continuously recognized.

As explained above, according to this embodiment, the following matters can be said.

When the second coupling portion 44 of the operation portion 24 is coupled with the first coupling portion 38 of the insertion portion 22 having the pigment-containing bag 72, an evidence that the insertion portion 22 has been coupled with the operation portion 24 can be left. Therefore, it is possible to easily recognize that a disposable portion (the insertion portion 22) has been already used in a surgical apparatus, e.g., the disposable endoscope 12. Additionally, even if the coupled state of the insertion portion 22 with respect to the operation portion 24 of the endoscope 22 is released, an indication that enables recognition that a used state is kept in the insertion portion 22, and hence erroneous reuse due to disappearance of the indication can be avoided. Therefore, whether the disposable portion has been used can be more assuredly recognized even after the insertion portion 22 is used and the coupled state with respect to the operation portion 24 is released in particular.

Further, even if cleaning or the like is performed in a state that the pigment-containing bag 72 is broken, the pigment partially still remains at any position in the insertion portion 22. Furthermore, the endoscope 12 having the insertion portion 22 on which the indication of the used state based on the attachment of the pigment is kept can make a strong impression on a patient and others. Therefore, since the use of such an endoscope 12 having the insertion portion 22 that has such the pigment attached thereto may possibly become a problem, the endoscope 12 can be hardly used for a new patient.

It is to be noted that the example that the judgment is made upon whether the connection has been achieved based on the change in color of the pigment after simply flowing the pigment in the second chamber 76 of the pigment-containing bag 72 into the first chamber 74 through the easy-peel portion 78 has been described herein. Additionally, it is also preferable to form the first chamber 74 of the pigment-containing bag 72 itself into appropriate characters or symbols such as “USED”. When such a configuration is adopted, whether the insertion portion 22 has been already connected with the operation portion 24 can be more assuredly recognized by raising characters or symbols in addition to changing the color.

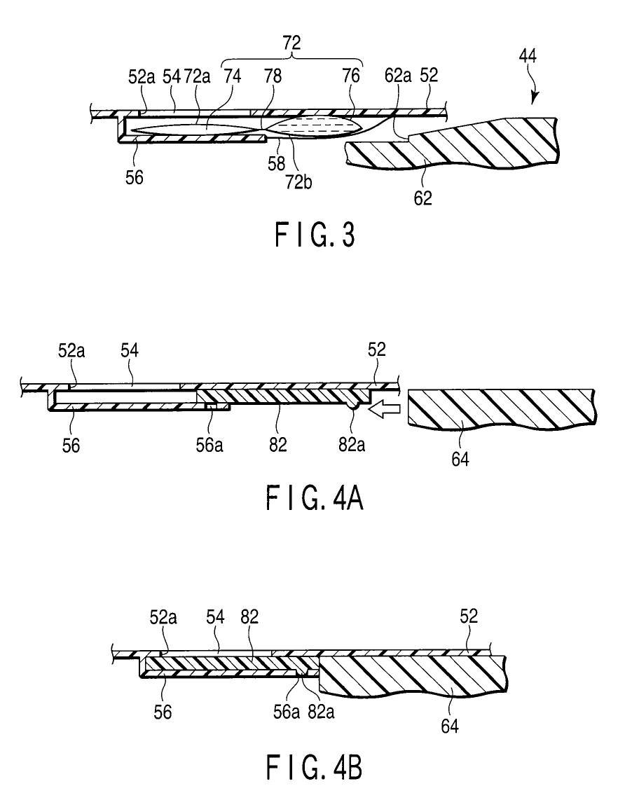

Further, although the pigment-containing bag 72 of the first coupling portion 38 of the insertion portion 22 is directly pushed by the second coupling portion 44 of the operation portion 24 in this example, it is also preferable to seal the accommodation portion 56 with the pigment-containing bag 72 being contained in a flexible sheet-like member 58 formed of, e.g., a polyolefin-based resin material, chloroethene, or PTFE depicted in FIG. 3.

In this case, one end of the sheet-like member 58 is fixed to an end part of the accommodation portion 56, and the other end of the sheet-like member 58 is fixed to the exterior frame 52 by an adhesive or the like. The sheet-like member 58 is formed to be hardly removed. When the sheet-like member 58 is removed, bored, or deformed, the state that the insertion portion 22 has been coupled with the operation portion 24 or the used state is confirmed, thereby enabling the recognition that the use should be avoided.

It is to be noted using a member that is plastically deformed or using a member that is destroyed in place of the flexible sheet-like member 58 is also preferable. Further, when the accommodation portion 56 is sealed by the sheet-like member 58 or the like, it is also preferable to use, e.g., a capsule that can be easily destroyed by pressing force caused at the time of coupling the operation portion 24 with the insertion portion 22 in place of using the pigment-containing bag 72. In this case, a pigment in the capsule diffuses in the accommodation portion 56 to cause the pigment to attach to the window portion 54, thus enabling a judgment on a change in color.

Furthermore, although the example where the window portion 54 is provided to the opening portion 52a of the exterior frame 52 has been explained, the window portion 54 does not have to be necessarily provided to the opening portion 52a, and a state that the communication with the outside of the accommodation portion 56 is achieved is also preferable.

Moreover, it is often the case that the appearance of the endoscope is black, but the appearance is not restricted to the black color, various colors are allowed, and a transparent color or the like is also allowed because the insertion portion 22 of the endoscope 12 according to this embodiment is of a disposable type. In this case, if the pigment is attached to the insertion portion 22, it, is preferable to select a pigment that is distinctive to an exterior color so that the attached pigment can be readily recognized.

Additionally, in this embodiment, the configuration that the endoscope 12 is divided into the insertion portion 22 and the operation portion 24 has been described, but a configuration that the insertion portion 22 and the operation portion 24 of the endoscope 12 are integrated can also obtain the same functions and effects. That is, in this embodiment, the configuration that the pigment-containing bag 72 is provided in the first coupling portion 38 of the insertion portion 22 has been described, but it is also preferable to provide the same configuration as the first coupling portion 38 to the connector 48 serving as the first coupling portion of the endoscope (the first medical equipment as a disposable part) 12 and provide the same configuration as the second coupling portion 44 to the light source (the second medical equipment as a reusable part) 14 serving as the second coupling portion, for example. In this case, the endoscopic system 10 as a medical apparatus can obtain the same functions and effects as those explained above in the relationship between the connector 48 of the endoscope 12 and the light source 14.

It is to be noted that, in this embodiment, any one or combinations of the pigment-containing bag 72, the opening portion 52a, the window portion 54, and others constitute a part of an indicator (indication means), and any one or combinations of the engagement portion 62, the pigment-containing bag 72, the easy-peel portion 78, and others constitute a keeping portion (keeping means).

### Second Embodiment

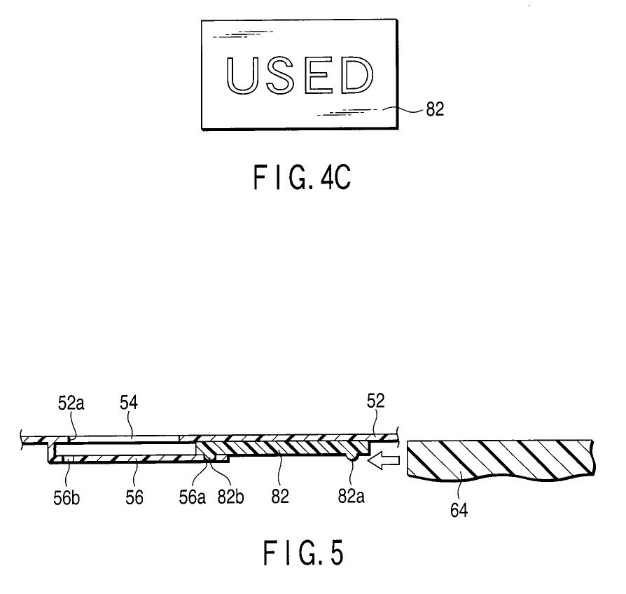

A second embodiment will now be described with reference to FIGS. 4A to 5. This embodiment is a modification of the first embodiment, and like reference numbers denote members equal to those described in the first embodiment, thereby omitting a detailed explanation.

As shown in FIGS. 4A and 4B, the first coupling portion 38 of the insertion portion 22 includes a flat plate 82 that can be readily put into the accommodation portion 56 and is hard to be removed from the same in place of the pigment-containing bag 72. For example, a concave portion 56a is formed in the accommodation portion 56, and a protrusion 82a is formed on the flat plate 82. Therefore, when the flat plate 82 is slid with respect to the accommodation portion 56, the protrusion 82a of the flat plate 82 engages with the concave portion 56a of the accommodation portion 56. Therefore, the flat plate 82 can be easily put into the accommodation portion 56, and the flat plate 82 that is once accommodated in the accommodation portion 56 is hard to be taken out. It is to be noted that a word, e.g., “USED” is provided on the flat plate 82 by printing or the like as shown in FIG. 4C.

An engagement portion 64 of the second coupling portion 44 of the operation portion 24 has a shape different from that of the engagement portion 62 described in the first embodiment, and it is formed to press an end of the flat plate 82 when putting the flat plate 82 into the accommodation portion 56.

A function of the endoscope 12 according to this embodiment will now be described.

As shown in FIG. 4A, when using the endoscope 12, the first coupling portion 38 of the insertion portion 22 is set to face the second coupling portion 44 of the operation portion 24. At this time, the flat plate 82 is placed at a position depicted in FIG. 4A. Therefore, a color of the wall surface of the accommodation portion 56 can be visually confirmed through the window portion 54.

Moreover, the first coupling portion 38 of the insertion portion 22 is coupled with the second coupling portion 44 of the operation portion 24 in a predetermined state. At this time, the end part of the flat plate 82 is pressed by the engagement portion 64 of the second coupling portion 44 of the operation portion 24. Therefore, the flat plate 82 is slid by this pressure, whereby the flat plate 82 is accommodated in the accommodation portion 56 as depicted in FIG. 4B. Therefore, the word “USED” on the flat plate 82 can be visually confirmed through the window portion 54.

Therefore, it is recognized that the disposable insertion portion 22 is coupled with the reusable operation portion 24. The endoscope 12 in this state is used for, e.g., a living tissue.

Additionally, after the use of the endoscope 12, the insertion portion 22 is separated from the operation portion 24. At this time, the state that the flat plate 82 is accommodated in the accommodation portion 56 is kept. Therefore, even if the insertion portion 22 is decoupled from the operation portion 24, the state that the insertion portion 22 is coupled with the operation portion 24 is continuously confirmed.

It is to be noted that the example where the single concave portion 56a of the accommodation portion 56 and the single protrusion 82a of the flat plate 82 are provided as depicted in FIGS. 4A and 4B has been described in this embodiment, but a configuration that another protrusion 82b of the flat plate 82 engages with the concave portion 56a of the accommodation portion 56 as shown in FIG. 5 is also preferable. Then, it is possible to prevent the flat plate 82 from being accommodated in the accommodation portion 56 when the flat plate 82 is slid before the engagement of the insertion portion 22 and the operation portion 24. Further, when the flat plate 82 has the other protrusion 82b in this manner, the other protrusion 82b of the flat plate 82 engages with the other concave portion 52b of the accommodation portion 56 at the time of coupling the insertion portion 22 with the operation portion 24.

Furthermore, in this embodiment, any one or combinations of the flat plate 82, the opening portion 52a, the window portion 54, and others constitute an indicator (indication means), and any one or combinations of the concave portion 56a (and the concave portion 56b), the protrusion 82a (and the protrusion 82b), the first coupling portion 38, the second coupling portion 44, the engagement portion 62, and others constitute a keeping portion (keeping means).

### Third Embodiment

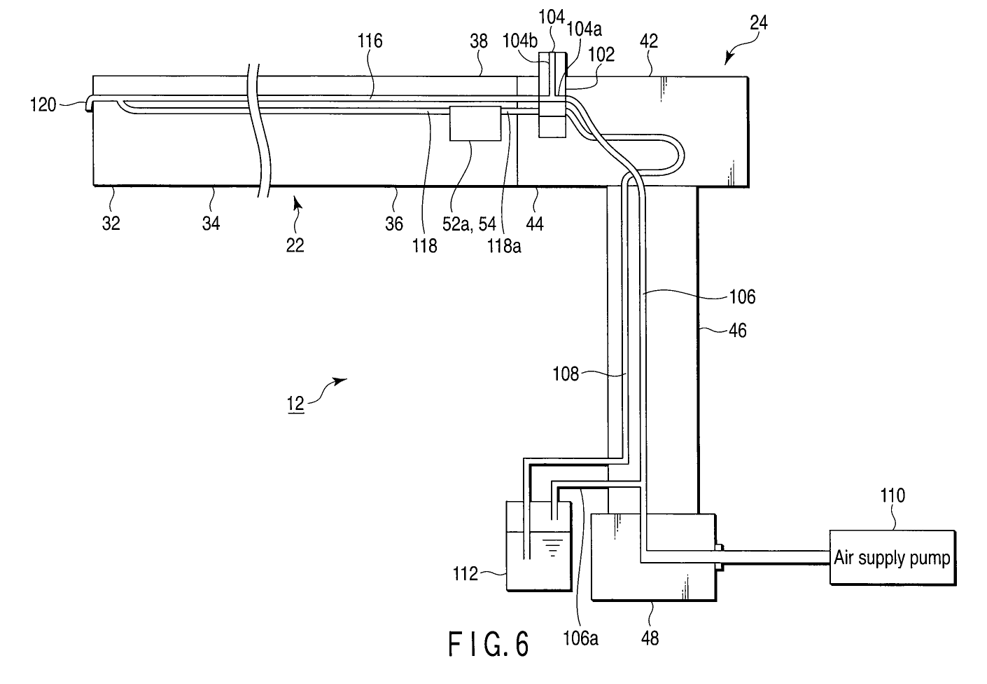

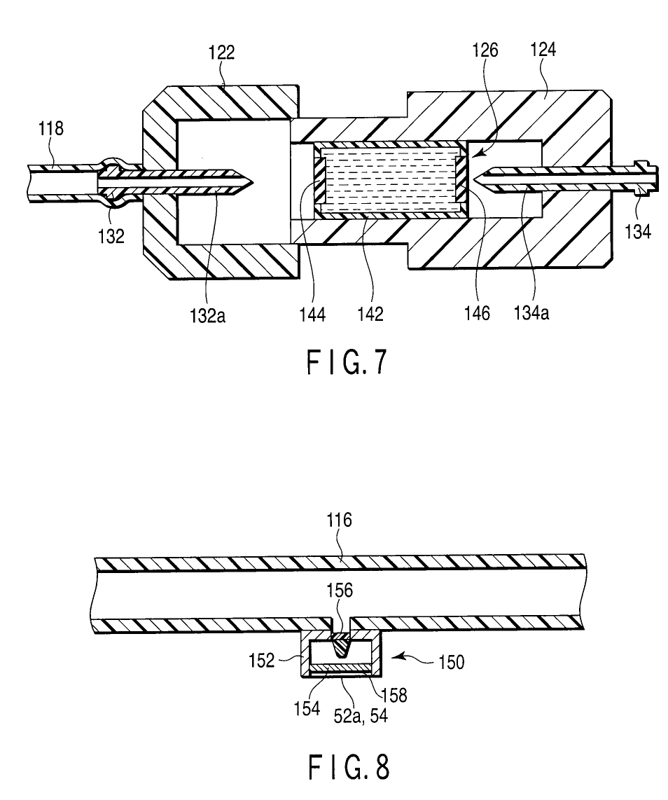

A third embodiment will now be described with reference to FIGS. 6 and 7. This embodiment is a modification of the first embodiment, and like reference numbers denote members equal to those described in the first embodiment, thereby omitting a detailed description thereof.

As shown in FIG. 6, a cylindrical air supply/water supply button arrangement portion 102 is formed in the operation portion main body 42 of the endoscope 12. An air supply/water supply button 104 that can be pushed in an axial direction with respect to the air supply/water supply button arrangement portion 102 is arranged on the air supply/water supply button arrangement portion 102. It is to be noted that a communication hole 104a and a leak hole 104b communicating with an operation-portion-side air supply tube 106 and an insertion-portion-side air supply tube 116 which will be described later are formed in the air supply/water supply button 104.

One end of each of the operation-portion-side air supply tube 106 and an operation-portion-side water supply tube 108 is connected with the air supply/water supply arrangement portion 102. Each of the operation-portion-side air supply tube 106 and the operation-portion-side water supply tube 108 is arranged in the universal cable 46. Further, an air supply pump 110 is connected with the other end of the operation-portion-side air supply tube 106 through the connector 48 in addition to the universal cable 46. A water supply bottle 112 is connected with the other end of the operation-portion-side water supply tube 108 through the universal cable 46. A branch tube 106a of the operation-portion-side air supply tube 106 is further connected with the water supply bottle 112. It is to be noted that the water supply bottle 112 is filled with a liquid such as a normal saline solution, and the other end of the operation-portion-side water supply tube 108 is provided in the liquid. On the other hand, an end part of the branch tube 106a is provided in a space in the water supply bottle 112.

In an insertion portion 22 of the endoscope 12, the insertion-portion-side air supply tube 116 and an insertion-portion-side water supply tube 118 are arranged separately from the operation-portion-side air supply tube 106 and the operation-portion-side water supply tube 108 of the operation portion 24. The insertion-portion-side air supply tube 116 and the insertion-portion-side water supply tube 118 are coupled with each other at a distal end portion of the insertion portion 22. An air supply/water supply nozzle 120 is arranged at a distal end portion of the insertion-portion-side air supply tube 116 and the insertion-portion-side water supply tube 118. That is, the air supply/water supply nozzle 120 is arranged to protrude from a distal end surface of the distal end hard portion 32.

Further, as explained above, when the air supply/water supply button 104 is operated, a communication state between the operation-portion-side air supply tube 106 and the insertion-portion-side air supply tube 116 is switched, and a communication state between the operation-portion-side water supply tube 108 and the insertion-portion-side water supply tube 118 is switched.

Here, when the leak hole 104b is opened, air supplied from the operation-portion-side air supply tube 106 is discharged to the outside from the leak hole 104b of the air supply/water supply button 104. When the leak hole 104b of the air supply/water supply button 104 is closed with a finger, the air supplied from the operation-portion-side air supply tube 106 is fed to the insertion-portion-side air supply tube 116 through the communication hole 104a to be discharged from the air supply/water supply nozzle 120.

When the air supply/water supply button 104 is pushed with the leak hole 104b being closed with a finger, the communication between the operation-portion-side air supply tube 106 and the insertion-portion-side air supply tube 116 is blocked, and the operation-portion-side water supply tube 108 and the insertion-portion-side water supply tube 118 communicate with each other through the communication hole 104a. Air flows into the water supply bottle 112 from the air supply pump 110 via the operation-portion-side air supply tube 106 and the branch tube 106a. Thus, pressure is applied to the liquid in the water supply bottle 112. Accordingly, the liquid in the water supply bottle 112 is supplied to the insertion-portion-side water supply tube 118 from the operation-portion-side water supply tube 108 to be discharged from the air supply/water supply nozzle 120.

The first coupling portion 38 of the insertion portion 22 is arranged in the middle of the insertion-portion-side water supply tube 118 arranged from the air supply/water supply button arrangement portion 102 toward the distal end side of the insertion portion 22.

As shown in FIGS. 6 and 7, the first coupling portion 38 includes a mechanism that enables coupling with the second coupling portion 44 of the operation portion 24, a fixing portion 122, a slide portion 124 slidably coupled with the fixing portion 122, and a pigment-containing capsule 126 arranged in the fixing portion 122. The fixing portion 122 and the slide portion 124 are lucidly formed to enable visually confirming the pigment-containing capsule 126 through the opening portion 52a of the exterior frame 52 or the window portion 54.

As a pigment in the pigment-containing capsule 126, it is possible to use a pigment whose trace can be left after use like the pigment described in each of the foregoing embodiments or a pigment whose trace cannot be left when a liquid such as wafer is caused to flow as different from the above-described pigment. Such a pigment may be supplied to the inside of a body in some cases, and hence one that meets criteria such as a Japanese Pharmaceutical Excipients or Japanese Standards of Food Additives is used.

The fixing portion 122 is formed into a bottomed cylindrical shape coupled with the first coupling portion 38. A first water supply tube 132 having a needle 132a is arranged on a bottom part of the fixing portion 122.

On the other hand, the slide portion 124 is formed into a bottomed cylindrical shape coupled with the fixing portion 122. A second water supply tube 134 having a needle 134a is arranged on a bottom part of the slide portion 124. Furthermore, the pigment-containing capsule 126 is arranged in the slide portion 124.

The pigment-containing capsule 126 includes a transparent cylindrical portion 142 and rubber stoppers 144 and 146 arranged at both ends of the cylindrical portion 142. Moreover, the cylindrical portion 142 of the pigment-containing capsule 126 is filled with a pigment and sealed by the rubber stoppers 144 and 146. Additionally, the needle 132a of the first water supply tube 132 of the fixing portion 122 faces one rubber stopper 144 of the pigment-containing capsule 126. The needle 134a of the second water supply tube 134 faces the other rubber stopper 146 of the pigment-containing capsule 126.

It is to be noted that, when coupling the first coupling portion 38 of the insertion portion 22 with the second coupling portion 44 of the operation portion 24, the second water supply tube 134 may be directly connected with the air supply/water supply button arrangement portion 102 or may be connected with the air supply/water supply button arrangement portion 102 through the tube 118a (see FIG. 6). On the other hand, the insertion-portion-side water supply tube 118 is fixed to the first water supply tube 132.

A function of the endoscope 12 according to this embodiment will now be described.

When coupling the first coupling portion 38 of the insertion portion 22 with the second coupling portion 44 of the operation portion 24, the slide portion 124 is pushed and moved with respect to the fixing portion 122. Further, when pushing force is applied to each of the needle 132a of the first water supply tube 132 and the needle 134a of the second water supply tube 134, the rubber stoppers 144 and 146 of the pigment-containing capsule 126 are burst. Therefore, the needle 132a of the first water supply tube 132 and the needle 134a of the second water supply tube 134 are inserted into the cylindrical portion 142.

In this state, the air supply/water supply button 104 is operated to flow the liquid from the water supply bottle 112 toward the first water supply tube 132 of the fixing portion 122 through the operation-portion-side water supply tube 108, the second water supply tube 134 of the slide portion 124, and the cylindrical portion 142 of the pigment-containing capsule 126. Then, the pigment in the cylindrical portion 142 of the pigment-containing capsule 126 flows out together with the liquid. Therefore, the pigment in the cylindrical portion 142 is gradually thinned. Further, although almost all of the pigment is eventually removed from the cylindrical portion 142 but, in case of the pigment whose trace remains, judging whether the pigment is present through the opening portion 52a of the exterior frame 52 or the window portion 54 enables recognition of whether the insertion portion 22 has been already used. Furthermore, in case of the pigment whose trace does not remain at all after flowing out, judging whether the pigment is present through the opening portion 52a of the exterior frame 52 or the window portion 54 enables recognition of whether the insertion portion 22 has been already used.

Moreover, even if the first coupling portion 38 of the insertion portion 22 is decoupled from the second coupling portion 44 of the operation portion 24, a state that the pigment-containing capsule 126 is arranged at the opening portion 52a of the exterior frame 52 or the window portion 54 is kept since the fixing portion 122 and the slide portion 124 are provided on the insertion portion 22 side.

It is to be noted that, in the embodiment using the pigment whose trace remains after supplying water, a configuration that a non-illustrated color comparison table is fixed at an edge part of the opening portion 52a of the exterior frame 52 is preferable.

As explained above, according to this embodiment, the following matters can be said.

After supplying the water from the water supply bottle 112 into the pigment-containing capsule 126 through the operation-portion-side water supply tube 108, the pigment partially leaves a trace or the pigment in the pigment-containing capsule 126 is caused to flow out to run out, whereby a state that the fluid such as a normal saline solution has been actually caused to flow to the operation-portion-side water supply tube 108 and the insertion-portion-side water supply tube 118 of the endoscope 12, i.e., a state that the endoscope 12 has been used, can be recognized. Furthermore, a state which enables confirming that the endoscope 12 has been already used can be kept.

It is to be noted that, in this embodiment, any one or combinations of the pigment-containing capsule 126, the opening portion 52a, the window portion 54, and others constitute an indicator (indication means), and any one or combinations of the first coupling portion 38, the second coupling portion 44, the opening portion 52a, the window portion 54, and others constitute a keeping portion (keeping means).

### Fourth Embodiment

A fourth embodiment will now be described with reference to FIG. 8. This embodiment is a modification of the third embodiment, and like reference numbers denote members equal to those described in the third embodiment, thereby omitting a detailed explanation thereof.

Although not shown, an oxygen indicator (a notification portion or indication means and a keeping portion or keeping means) 150 is fixed to the opening portion 52a of the exterior frame 52 or the window portion 54 of the first coupling portion 38 of the insertion portion 22. The oxygen indicator 150 is also fixed to the insertion-portion-side air supply tube 116.

As shown in FIG. 8, the oxygen indicator 150 includes a frame body 152, an oxygen barrier film 154 arranged on the frame body 152, a check valve 156 arranged between the inside of the frame body 152 and the insertion-portion-side air supply tube 116, and a film-like or plate-like pigment 158. The check valve 156 allows the passage of gas from the insertion-portion-side air supply tube 116 but avoids the passage of the gas from an opposite direction, i.e., the oxygen indicator 150 side. Additionally, a part of the frame body 152 substitutes for the window portion 54 arranged on the opening portion 52a of the exterior frame 52 to prevent gas (oxygen) from entering. The pigment 158 contains a redox pigment and reacts with oxygen to change its color. As a material of the oxygen barrier film, any material having transparency such as an ethylene-vinylalcohol copolymer (EVOH) resin material or a polyvinylidene-chloride (PVDC) resin can be preferably utilized.

Therefore, when air enters the frame body 152 from the air supply pump 110 through the operation-portion-side air supply tube 106, the insertion-portion-side air supply tube 116, and the check valve 156 of the oxygen indicator 150, oxygen contained in the air reacts with the pigment 158. This can be observed through the oxygen barrier film 154. Further, the pigment 158 keeps a state after such a reaction. Therefore, visually confirming the state of the pigment 158 through the oxygen barrier film 154 enables recognition of whether the insertion portion 22 has been already used.

It is to be noted that, when the pigment 158 is to be replaced, the new pigment 158 may highly possibly react with oxygen, and hence the pigment 158 must be replaced together with the oxygen indicator 150. However, when replacement is carried out in this manner, the insertion-portion-side air supply tube 116 must be replaced in addition to the oxygen indicator 150. In this case, since the insertion portion 22 must be disassembled, reuse of the insertion portion 22 is avoided.

### Fifth Embodiment

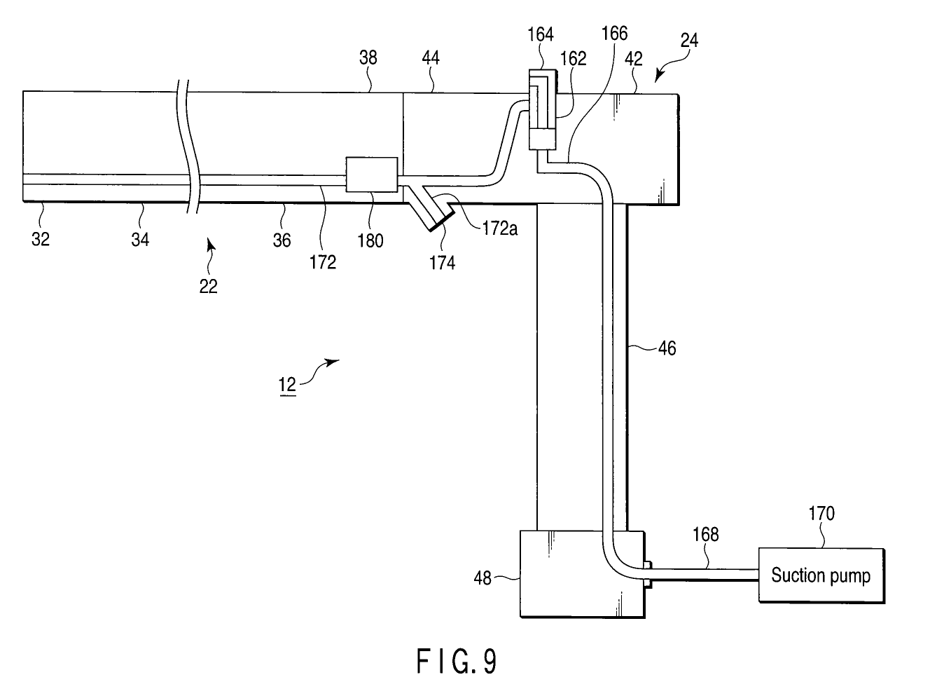

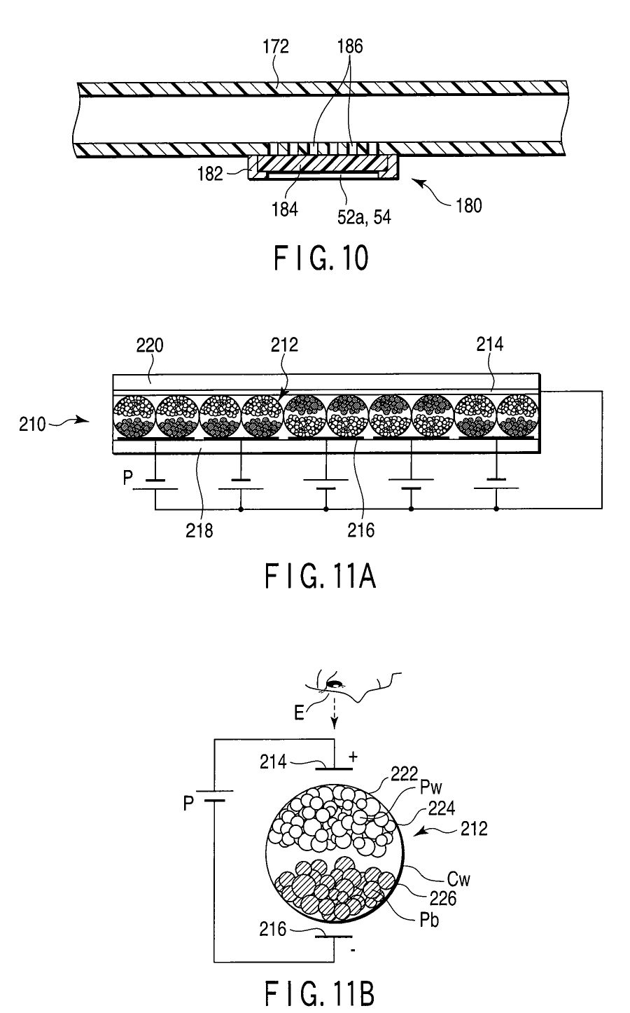

A fifth embodiment will now be described with reference to FIGS. 9 and 10. This embodiment is a modification of the third embodiment, and like reference numbers denote members equal to those described in the third embodiment, thereby omitting a detailed explanation thereof.

As shown in FIG. 9, a cylindrical suction button arrangement portion 162 is formed in the operation portion main body 42 of the endoscope 12. A suction button 164 that can be pushed in an axial direction with respect to the suction button arrangement portion 162 is arranged on the suction button arrangement portion 162.

One end of an operation-portion-side suction tube 166 is connected with the suction button arrangement portion 162. The operation-portion-side suction tube 166 is arranged in the universal cable 46. Further, the other end of the operation-portion-side suction tube 166 is fixed to the connector 48 through the universal cable 46. Furthermore, one end of a suction-pump-side tube 168 is arranged in the connector 48. The other end of the suction-pump-side tube 168 is connected with a suction pump 170. The suction pump 170 includes a suction bottle (not shown) in which a suctioned substance is arranged.

An insertion-portion-side suction tube 172 of the insertion portion 22 is arranged in the insertion portion 22 of the endoscope 12 separately from the operation-portion-side suction tube 166 of the operation portion 24. A distal end part of the insertion-portion-side suction tube 172 is opened at the distal end of the distal end hard portion 32.

Moreover, a branch tube 172a communicating with the insertion-portion-side suction tube 172 is arranged at a proximal end portion of the insertion portion 22. An end portion of the branch tube 172a is arranged in a forceps opening portion 174. A non-illustrated forceps stopper is arranged in the forceps opening portion 174 so that forceps can be arranged in a state that the insertion-portion-side suction tube 172 is sealed.

As shown in FIGS. 9 and 10, a pH indicator (a notification portion or indication means) 180 is fixed to the opening portion 52a of the exterior frame 52 or the window portion 54 of the first coupling portion 38 of the insertion portion 22. The pH indicator 180 is also fixed to the insertion-portion-side suction tube 172.

As shown in FIG. 10, the pH indicator 180 includes a frame body 182 and a moisture holding substrate (a keeping portion or keeping means) 184 such as dried gel arranged in the frame body 182. When a part of the frame body 182 serves as the window portion 54 arranged on the opening portion 52a of the exterior frame 52. The moisture holding substrate 184 contains a pH indicator that undergoes a color change in a weak acidic to strong acidic region or a weak acidic to weak basic region. In the insertion-portion-side suction tube 172, a plurality of opening portions 186 through which a suctioned substance comes into contact with the moisture holding substrate 184 are formed at positions facing the frame body 182. Further, the moisture holding substrate 184 is formed into a configuration that it cannot be removed from the frame body 182 since it is fixed to the frame body 182, for example. Furthermore, the frame body 182 is also formed into a shape in which the moisture holding substrate 184 cannot be newly put.

It is to be noted that, as the pH indicator, methyl orange, methyl red, or litmus is used. Moreover, for example, thymol blue, litmus, bromthymol blue, or phenol red is used for an upper endoscope and a lower endoscope. It is to be noted that congo red, phenol red, or the like is used for the upper endoscope and methyl yellow, methyl orange, methyl red, or the like is used for the lower endoscope, but any indicator which undergoes a color change in a weak acidic to strong acidic region can be preferably used for the upper endoscope, and any indicator which undergoes a color change in a weak acidic to weak basic region can be used for the lower endoscope.

Therefore, when the suction button 164 is operated to perform suction in a state that the endoscope 12 is inserted in, e.g., a body cavity, a suctioned substance comes into contact with the moisture holding substrate 184 of the frame body 182 through the opening portion 186 of the insertion-portion-side suction tube 172. Additionally, a color of the pH indicator contained in the moisture holding substrate 184 changes in accordance with properties of the suctioned substance. This can be observed through the opening portion 52a of the exterior frame 52 or the window portion 54. Further, the moisture holding substrate 184 containing the pH indicator keeps a state after the color has changed in this manner. Therefore, visually confirming the state of the moisture holding substrate 184 through the window portion 54 enables recognition of whether the insertion portion 22 has been already used.

It is to be noted that the configuration using the oxygen indicator 150 has been explained in the fourth embodiment whilst the configuration using the pH indicator 180 has been explained in the fifth embodiment, but using, e.g., a water leakage detection sticker (not shown) which is called “water indicator” is also preferable. For example, a red or pink dot pattern is printed on a surface of the water leakage detection sticker (an indicator or indication means and a keeping portion or keeping means) and the dot pattern is dissolved to bleed when getting wet with water, and hence a history recording that the pattern got wet with water in the past can be recognized even after drying. Alternatively, an indicator which is originally substantially white but gets red when coming into contact with water and keeps the color even after drying is also used.

### Sixth Embodiment

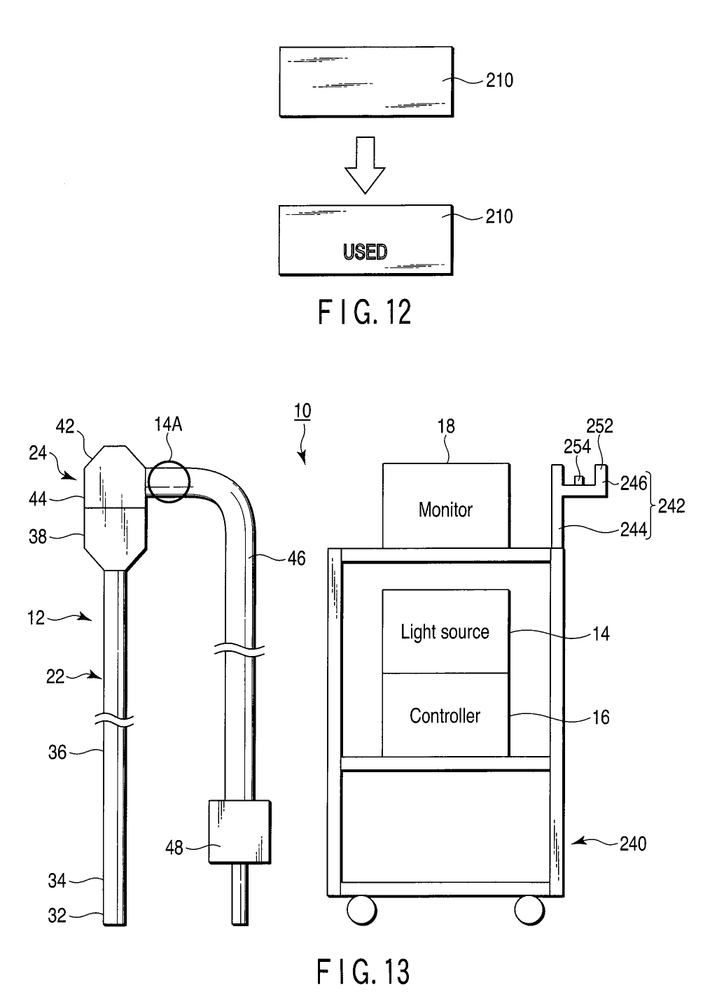

A sixth embodiment will now be described with reference to FIGS. 11A to 12. This embodiment is a modification of the first embodiment, and like reference numbers denote members equal to those described in the first embodiment, thereby omitting a detailed description thereof.

A digital paper (an indicator or indication means and a keeping portion and keeping means) 210 is embedded in the first coupling portion 38 of the insertion portion 22. The digital paper (electronic paper) 210 is fixed to the opening portion 52a of the exterior frame 52 or the window portion 54 of the first coupling portion 38 of the insertion portion 22.

For the digital paper 210, for example, there is adopted a microcapsule electrophoretic scheme that uses charged white and black particles in a transparent microcapsule with which a substrate surface is coated, applies a voltage P, and thereby moves pigment particle to effect indication.

As shown in FIG. 11A, the microcapsule type digital paper 210 includes many microcapsules 212, a transparent electrode (ITO) 214 and a back plate 216 which are utilized to arrange these microcapsules 212 in one layer, a polyimide film 218 having printed circuits printed on both surfaces, and a PET film 220. As shown in FIG. 11B, the microcapsule 212 includes a capsule wall 222, and black fine particles 224 and white fine particles 226 which are arranged in the capsule wall 222. The transparent electrode 214 and the back plate 216 are arranged to enable changing polarities thereof.

It is to be noted that a switch of the digital paper 210 is not shown, but it is turned on when the first coupling portion 38 of the insertion portion 22 is coupled with the second coupling portion 44 of the operation portion 24 for the first time. Further, even after the first coupling portion 38 is decoupled from the second coupling portion 44, the ON state of the switch is kept when the first coupling portion 38 is plastically deformed, for example. Furthermore, for a power supply (a keeping portion or keeping means) of the digital paper 210, it is preferable to use a battery arranged in the first coupling portion 38 of the insertion portion 22, electric power that is supplied to a non-illustrated imaging element provided in the distal end hard portion 32 when the controller 16 is connected with the endoscope 12, or electric power that is supplied to an imaging element (not shown) provided in the distal end hard portion 32 when the insertion portion 22 is connected with the operation portion 24.

Therefore, when the transparent electrode 214 on an observation side of a user of the endoscope 12 denoted by reference character E depicted in FIG. 11B is charged to have a positive polarity, the white fine particles 226 charged to have a negative polarity move toward the electrode. The black fine particles 224 charged to have the positive polarity move toward the back plate 216. Therefore, the microcapsule 212 is visually confirmed as being black.

Moreover, when the microcapsules 212 are controlled in the entire digital paper 210, characters “USED” are shown on the digital paper 210 in this embodiment as shown in FIG. 12. Additionally, in this embodiment, when the characters “USED” are once shown, the switched is turned on to keep the indication. That is, when the first coupling portion 38 of the insertion portion 22 is coupled with the second coupling portion 44 of the operation portion 24, the switch is kept in the ON state due to the coupling, and means for turning off the switch is not provided. Further, since such digital paper 210 can effect the indication with low electric power, the battery slowly runs out, whereby the indication of the characters “USED” can be kept for a longer period of time. Therefore, the state that the insertion portion 22 has been used can be recognized for a long time.

It is to be noted that the description has been given as to the example that the characters “USED” are shown alone in this embodiment, but it is also preferable to effect on the digital paper 210 the indication of various kinds of information, e.g., information indicating that the endoscope 12 is normal or abnormal when the insertion portion 22 and the operation portion 24 of the endoscope 12 are coupled with each other (including information indicative of an abnormal part in case of abnormality) or information indicating that the endoscope 12 is normal or abnormal when the endoscope 12 is connected with the light source 14.

Furthermore, although the microcapsule electrophorestic device is taken as the example of the digital paper 210 in the foregoing embodiment, various kinds of devices such as a flexible organic EL device, a liquid crystal display device, a twist ball display device, a powder transfer display device, or a thermal display device can be utilized.

### Seventh Embodiment

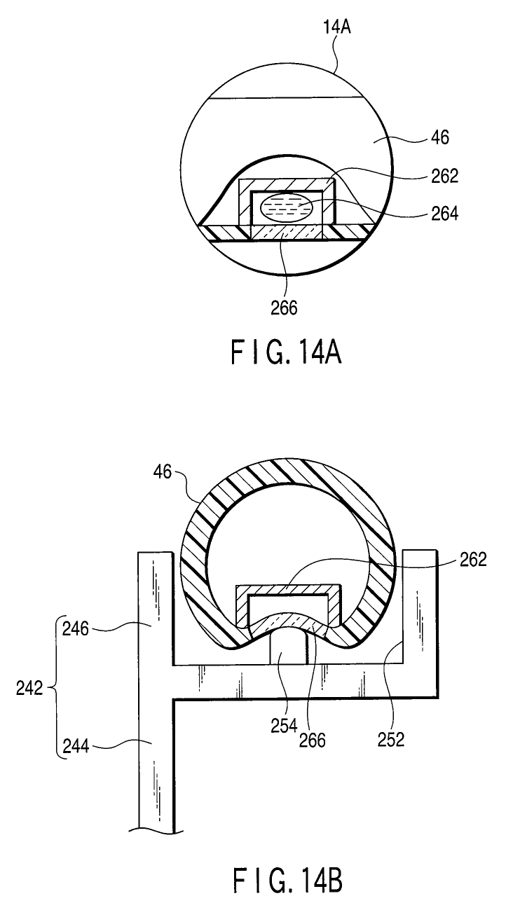

A seventh embodiment will now be described with reference to FIGS. 13 to 14B. This embodiment is a modification of the first embodiment, and like reference numbers denote members equal to those described in the first embodiment, thereby omitting a detailed description thereof.

As shown in FIG. 13, in the endoscopic system (a medical apparatus) 10, peripheral devices of the endoscope (a first medical equipment) 12 such as the light source 14 or the controller 16 are arranged on a trolley (a second medical equipment) 240 at the time of use. A scope hanger 242 on which the endoscope 12 is put is arranged on the trolley 240. The scope hanger (a coupling portion) 242 includes a column support 244 whose protruding length from the trolley 240 can be changed and a holding portion 246 arranged at a top portion of the support column 244.

The holding portion 246 includes a substantially-U-like concave portion 252. The concave portion 252 is used for hooking the operation portion main body 42 and hooking the universal cable 46 extended from the operation portion main body 42. Furthermore, a protrusion 254 is formed on the concave portion 252. The protrusion 254 is formed at a position at which a later-described transparent window 266 is pushed when the endoscope 12 is put on the scope hanger 242.

On the other hand, as shown in FIG. 14A, at a root portion of the universal cable 46 of the endoscope 12 (a part of the universal cable 46 that is designated by reference character 14A in FIG. 13 as a part close to the operation portion main body 42), a pigment-containing portion 262 is formed into a concave shape. For example, a capsule 264 containing a pigment is arranged in the pigment-containing portion 262. Moreover, a clear and flexible transparent window 266 is arranged and sealed on the pigment accommodation portion 262 with the capsule 264 being put in the portion. It is to be noted that a space formed between the transparent window 266 and the pigment accommodation portion 262 is formed to be very narrow. The flexibility of the transparent window 266 is formed in such a manner that a volume of a space formed between the transparent window 266 and the pigment accommodation portion 262 is greatly reduced as shown in FIG. 14B when the endoscope 12 is put on the scope hanger 242.

A function of the endoscopic system 10 according to the embodiment will now be described.

Before using the endoscope 12, as shown in FIG. 14A, the capsule 264 in the pigment-containing portion 262 is filled with a pigment as shown in FIG. 14A. Additionally, the endoscope 12 is put on the scope hanger 242 before using the endoscope 12 or in order to use any other equipment. At this time, the transparent window 266 of the endoscope 12 is pressed against the protrusion 254 of the scope hanger 242 due to the gravity of the endoscope 12 itself. Therefore, as shown in FIG. 14B, the capsule 264 in the pigment accommodation portion 262 receives a pressure to be destroyed. Accordingly, the pigment in the capsule 264 flows out into the space between the pigment accommodation portion 262 and the transparent window 266. Therefore, it is possible to readily recognize that the pigment has adhered to the transparent window 266 and the capsule 264 has been destroyed through the transparent window 266 and also recognize a change in color that can be visually confirmed through the transparent window 266.

Here, the transparent window 266 cannot be removed. Alternatively, since an unnatural repair mark or the like remains when the transparent window 266 is removed, even if time elapses after use of the endoscope 12, whether the preparation for using the endoscope 12 has been made or whether the endoscope 12 has been used can be recognized.

It is to be noted that the description has been given as to the example where the pigment accommodation portion 262 is provided at the root portion (the part denoted by reference character 14A in FIG. 13) of the universal cable 46 with respect to the operation portion main body 42, but it is also preferable to provide the pigment accommodation portion 262 at any position, e.g., the operation portion main body 42 itself to which the gravity is applied in a state that the transparent window 266 is in contact with the protrusion 254 of the scope hanger 242.

Further, in this embodiment, the capsule 264 constitutes an indicator or indication means, and the pigment-containing portion 262 and the transparent window 266 constitute a keeping portion or keeping means.

Although the several embodiments have been specifically described with reference to the drawings, the present invention is not restricted to the foregoing embodiments, and it includes all embodiments carried out without departing from the scope thereof.

According to the preceding embodiments, there can be provided the medical apparatus that can readily determine that the medical equipments were coupled with each other in the past or that the medical equipments were used in the past while being coupled with each other even after decoupling.

What is claimed is:

1. A medical apparatus comprising:
a first medical equipment used for performing a medical action with respect to an object;
a second medical equipment including a coupling portion attachable to/detachable from the first medical equipment;
an indicator attached to the first medical equipment and/or second medical equipment and configured to indicate that the first and second medical equipments are coupled with each other, simultaneous with coupling between the first and second medical equipments; and
a keeping portion which irreversibly keeps a state indicated by the indicator.

2. The medical apparatus according to claim 1, wherein the indicator indicates that the first medical equipment has never been coupled with the second medical equipment.

3. The medical apparatus according to claim 2, wherein the indicator switches from an indication that that the first medical equipment has never been coupled with the second medical equipment to an indication that the first and second medical equipments are coupled, by utilization of a force with which the first and second equipments are coupled.

4. The medical apparatus according to claim 2, wherein the indicator indicates that the first medical equipment and/or the second medical equipment were used before.

5. The medical apparatus according to claim 1, wherein the indicator indicates that the first medical equipment and/or the second medical equipment were used before.

6. A medical apparatus comprising:
a first medical equipment used for performing a medical action with respect to an object;
a second medical equipment including a coupling portion attachable to/detachable from the first medical equipment;
indication means, attached to the first medical equipment and/or second medical equipment, for indicating that the first and second medical equipments are coupled with each other simultaneous with coupling between the first and second medical equipments; and
keeping means for irreversibly keeping a state indicated by the indication means.

7. The medical apparatus according to claim 6, wherein the indication means indicates that the first medical equipment has never been coupled with the second medical equipment.

8. The medical apparatus according to claim 7, wherein the indication means switches from an indication that the first medical equipment has never been coupled with the second medical equipment to an indication that the first and second medical equipments are coupled, by utilization of a force with which the first and second equipments are coupled.

9. The medical apparatus according to claim 7, wherein the indication means indicates that the first medical equipment and/or the second medical equipment were used before.

10. The medical apparatus according to claim 6, wherein the indication means indicates that the first medical equipment and/or the second medical equipment were used before.
